(19) United States
(12) Patent Application Publication
(10) Pub. No.: US 2026/0262939 A1
(43) Pub. Date: Sep. 10, 2026

(54) TONOMETER FOR DETERMINING PROPERTIES OF EYE AND METHOD THEREOF

(71) Applicant: ICARE FINLAND OY, Vantaa (FI)

(72) Inventors: Andrei Plaian, Noventa Padovana, Pd (IT); Iina Korkka, Hyvinkää (FI)

(73) Assignee: ICARE FINLAND OY, Vantaa (FI)

(21) Appl. No.: 19/473,622

(22) PCT Filed: Apr. 16, 2024
(86) PCT No.: PCT/FI2024/050174

§ 371 Date: Oct. 8, 2025

(30) Foreign Application Priority Data
Apr. 25, 2023 (FI) 20235458

(51) Int. Cl. A61B 3/16; A61B 3/10

(52) U.S. Cl. CPC A61B 3/165; A61B 3/1005

(57) ABSTRACT

A tonometer for determining properties of eye, tonometer includes execution unit with impact means arranged to apply, when in use, impact on cornea of eye; at least one measuring unit, first confocal chromatic sensor; controller connected to execution unit, measuring unit and first confocal chromatic sensor, wherein controller is configured, when in use, to use: execution unit to apply impact on cornea of eye using impact means; at least one measuring unit to determine intraocular pressure value of eye; and first confocal chromatic sensor to measure corneal thickness value of eye, wherein at least one measuring unit is velocity measurement unit, for measuring velocity profile of impact means, during impact, and controller is further configured to use measured velocity profile to determine intraocular pressure value of eye.

### TECHNICAL FIELD

The present disclosure relates to a tonometer for measuring properties of an eye. The present disclosure also relates to a method of measuring properties of an eye.

### BACKGROUND

Intraocular pressure (IOP) is a crucial factor to keep in check as elevated IOP is one of the most important risk factors for development and progression of glaucoma, a leading cause of irreversible blindness worldwide. Glaucoma is a group of eye diseases characterized by progressive damage to the optic nerve, often leading to vision loss and blindness if left untreated. Elevated IOP can cause damage to the optic nerve by compressing blood vessels and reducing the blood supply to the optic nerve.

Intraocular pressure (IOP) can be measured using various of techniques. As an example, a Goldmann applanation tonometer can be used to measure IOP. In said tonometer type the pressure is determined by measuring a force applied to a cornea by part of the tonometer. The measured force can be used to deduct IOP value. An alternative method is usage of re-bound tonometer. In re-bound tonometry a projectile is ejected towards cornea and change of speed of the projectile is measured. This change of speed (speed profile) can be used to determine what value for intraocular pressure. A problem with above methods is that the derived intraocular pressure value is a theoretical value based on assumptions of eye physiology. As an example, Goldmann applanation is based on assumptions related to thickness of the eye. Indeed a standard corneal thickness of 520 micrometres is assumed. If the assumptions are wrong, then the measurement results do not provide accurate values. In a similar manner when considering the re-bound tonometer reading, it might be inaccurate for users who's corneal properties are not as in underlying models or tests. These might lead to measurement errors in IOP values. Measurement errors in medical devices might lead to wrong diagnoses and might risk health of a patients.

Therefore, in light of the foregoing discussion, there exists a need to overcome the aforementioned drawbacks associated with conventional tonometers and methods of measuring the IOP using the conventional tonometers.

### SUMMARY

The present disclosure seeks to provide a tonometer for determining properties of an eye. The present disclosure also seeks to provide a method of determining properties of an eye. An aim of the present disclosure is to provide a solution that overcomes at least partially the problems encountered in prior art.

In one aspect, an embodiment of the present disclosure provides a tonometer for determining properties of an eye, the tonometer comprising
an execution unit comprising an impact means arranged to apply, when in use, an impact on a cornea of the eye;
at least one measuring unit;
a first confocal chromatic sensor;
a controller connected to the execution unit, the measuring unit and the first confocal chromatic sensor;
wherein the controller is configured, when in use, to use:
the execution unit to apply an impact on the cornea of the eye using the impact means,
the at least one measuring unit to determine an intraocular pressure value of an eye, and
the first confocal chromatic sensor to measure a corneal thickness value of the eye.

In another aspect, an embodiment of the present disclosure provides a method of measuring properties of an eye, the method comprising:
a) applying an impact on a cornea of the eye using an execution unit comprised in a tonometer, wherein the execution unit comprises an impact means arranged to apply, when in use, the impact on the cornea of the eye;
b) measuring an intraocular pressure value of the eye using at least one measuring unit comprised in the tonometer and;
c) measuring a corneal thickness value of the eye using at least one confocal chromatic sensor comprised in the tonometer.
Embodiments of the present disclosure substantially eliminate or at least partially address the aforementioned problems in the prior art, and enable precise detection of the intraocular pressure value of the eye and the corneal thickness value of the eye using the tonometer which is non-invasive.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a tonometer for measuring properties of an eye, in accordance with an embodiment of the present disclosure;

FIG. 2 is a block diagram illustrating a tonometer for measuring properties of an eye;

FIG. 3 is a graphical representation depicting oscillations in cornea with respect to time, in accordance with an embodiment of the present disclosure;

FIG. 4 is a flowchart depicting steps of a method for measuring properties of an eye, in accordance with an embodiment of the present disclosure; and

FIG. 5 is illustration of a tonometer, which is according to an embodiment based on air impulse.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, an embodiment of the present disclosure provides a tonometer for determining properties of an eye, the tonometer comprising
an execution unit comprising an impact means arranged to apply, when in use, an impact on a cornea of the eye, when in use;
at least one measuring unit;
a first confocal chromatic sensor;
a controller connected to the execution unit, the measuring unit and the first confocal chromatic sensor; Wherein the controller is configured to, when in use, to use:
the execution unit to apply an impact on the cornea of the eye using the impact means;
the at least one measuring unit to determine an intraocular pressure value of an eye; and
the first confocal chromatic sensor to measure a corneal thickness value of the eye.

The term “when in use” refer to using the tonometer. I.e., impact is applied to the cornea of the eye during the use of the tonometer and the controller uses the execution unit, the at least one measuring unit and the first confocal sensor when the tonometer is used to determine properties of the eye.

In another aspect, an embodiment of the present disclosure provides a method of measuring properties of an eye, the method comprising:
a) applying an impact on a cornea of the eye using an execution unit comprised in a tonometer, wherein the execution unit comprises an impact means arranged to apply, when in use, the impact on the cornea of the eye;
b) measuring an intraocular pressure value of the eye using at least one measuring unit comprised in the tonometer, and
c) measuring a corneal thickness value of the eye using at least one confocal chromatic sensor comprised in the tonometer.

The present disclosure provides the aforementioned tonometer for determining properties of an eye and a method of determining properties of an eye. One of the properties, which can be determined or measured with the tonometer, is an intraocular pressure of an eye. Another property, which can be determined or measured with disclosed tonometer, is a corneal thickness value. Further, the confocal chromatic sensor of the tonometer enables accurate and simultaneous determine of the intraocular pressure value of the eye and/or corneal thickness value of the eye. Beneficially, the corneal thickness value of the eye can be used to correct the intraocular pressure value of the eye derived from the measurements with at least one measuring unit. More beneficially, the tonometer of the present disclosure can measure a distance between a front part of the tonometer and the eye which can be used to correctly position the tonometer with respect to the eye to enable efficient examination of the eye. Indeed, the distance measurement can be used automatically to trigger measurement cycle or to indicate for the user to initiate the measurement cycle. More beneficially, the tonometer is compact in size, enables to determine two different properties of the eye with the same device (IOP and corneal thickness) and is easy to use. Further, the method of measuring the properties of the eye is easy to implement. Corneal thickness value and the intraocular pressure value of eye are preferably measured within short time interval from each other's such as within 0.1 sec, 0.2 sec, 0.3 sec, 0.4 sec, 0.5 sec, 0.6 sec, 0.7 sec, 0.8 sec, 0.9 sec, 1 sec, 5 sec, 10 sec, 30 sec, 1 min in order to have valid correction factor for intraocular pressure value.

The term “execution unit” as used herein refers to an arrangement that is used for applying a force on the cornea of the eye. The term “impact means” as used herein refers to a means that is used for impacting a surface. The impact means is arranged as part of the execution unit. In an example, the impact means is used for impacting the surface of the eye. Impact means can be for example a probe which is launched with the execution unit towards the eye. Alternatively, the impact means can be an air impulse i.e. relatively short duration of flow of air towards eye. It will be appreciated that the impact means is arranged to create the impact preferably on the cornea of the eye and without impacting the whole eye together or a volume of tissues surrounding the eye.

The term “measuring unit” as used herein refers to a component that is used for measuring intraocular pressure (IOP). The measuring unit might measure (or determine) the IOP by measuring a physical event and use those measurement results to derive the measurement result or value. As an example, a speed profile of the impact means (of a probe as an impact means) can be measured with the measuring unit. The speed profile can be used to determine IOP. As an example, if rebound of the impact means is rapid it might indicate larger IOP than if the rebound of the impact means is slower. Another example is measuring frequency of free oscillations on the cornea after impact. The frequency of the oscillations of the cornea is indicative of intraocular pressure (IOP) value of the cornea. Indeed, it has been observed that there is almost direct correlation between the frequency and IOP. As an example if frequency is 250 Hz the IOP value is 10 mmHg. The IOP increases as function of the frequency (or vice versa) linearly in such a way, that when frequency is 360 Hz, corresponding IOP value is 40 mmHg. Frequency value of 300 Hz corresponds to IOP value of about 20 mmHg. The correlation can be used, for example via equation or look up table to provide IOP value after determining the oscillation frequency.

The term “intraocular pressure” refers to a fluid pressure of the eye. Optionally, the measuring unit may apply different mathematical models, such as regression analysis or curve fitting, to the raw data to obtain the most accurate and reliable measurements of the internal pressure value of the eye. Optionally, the measuring unit may perform other functions, such as error correction, data storage, and data analysis. Notably, the IOP value of the eye is determined to maintain overall eye health and function. Optionally, the eye presents a mass-spring system, where the IOP value describes a spring constant. Optionally, when the cornea is displaced from its natural position by an external force, it vibrates as a damped harmonic oscillator with a frequency relative to the IOP. The IOP modulates the stiffness of the eye as well as the cornea so that the oscillation frequency increases with increasing IOP. It will be appreciated that the measurement of the IOP value enables a diagnosis and treatment of ocular hypertension which may lead to Glaucoma. Notably, a higher IOP value is indicative of an increased stiffness of the eyeball, and therefore an increased resonance frequency of the cornea.

Optionally, the impact means may be arranged partially within a hollow space of the loops (loops being arranged as coil to surround the impact means at least partly) of the measuring unit. The impact means in this example include magnetic portion, such as elongated magnetic body. In such a case, the impact means may move through the loops of the measuring unit. Moreover, the movement of the impact means inside the measuring unit produces an induced voltage in the measuring unit. This induced voltage is an indication of speed of the impact means. By determining a speed profile, it is possible to determine (measure) IOP value.

Optionally as discussed above, the impact means may be implemented as a probe. Optionally, the impact means is a magnetic elongated probe. The magnetic elongated probe may be partially arranged within the execution unit. It will be appreciated that the magnetic elongated probe has two opposite ends: the first end and the second end, such that the second end is arranged to be inside the execution unit, and the first end protrudes outside of the execution unit from an opening of the execution unit. The magnetic elongated probe also has a middle section between the first end and the second end. Optionally, the first end of the magnetic elongated probe is made from bio-compatible material and will collide with a surface of the object (such as the cornea of the eye) when in use. Beneficially, the first part being made of bio-compatible material enables the probe to function in intimate contact with living tissues of the eye, for example, causing minimal discomfort or pain. Notably, the bio-compatible material is free from carcinogenicity, toxicity, and is resistive to corrosion. Optionally, the execution unit comprises a set of electrically controllable loops (coils arranged to surround at least partly the magnetic elongated probe). As the electricity is applied to the loops it generates magnetic force which will eject the impact means towards a target (cornea of the eye).

Optionally the impact means is an air impulse having a first duration of time and a first force. Air impulse can be generated for example by having a pressured container having a nozzle controlled by a valve. The nozzle is directed towards eye and when the valve is opened for the first duration of time air will flow from the pressured container via the nozzle towards the eye. The movement of air will result to the first force which is proportional to pressure difference between ambient pressure and the pressured container, size of the nozzle and distance of the nozzle from the eye. An air impulse is generated thus by keeping the valve opened over the first duration of time. After the first period of time the air impulse is stopped. According to an embodiment the first force of the air impulse is stopped with a decay time that is shorter than the time period of the free corneal oscillation. This way we ensure that there is rapid enough stopping of the force impacting the eye surface. After stopping the air impulse force the eye starts to oscillate. Decay of stopping is preferably faster than a time period of the free corneal oscillation. Since typical frequencies vary between 100 Hz to 500 Hz the air impulse should be preferably stopped within 1/500 Hz=2 msec from full force to zero level. (by closing valve for example). Alternative way to implement air impulse can be for example using piston type of arrangement.

The term “confocal chromatic sensor” (CCS) as used herein refers to an instrument that works by focusing a beam of light with a wide band of wavelengths, usually white light, onto a target surface (such as the eye) using a high dispersion objective lens. Optionally, the beam of light is preferably oriented normally to the cornea. Advantageously, the confocal chromatic sensors possess the ability to tolerate a large inclination of an axis of the beam with respect to the normal direction of the measured surface (even tens of degrees). The confocal chromatic sensor can be used to measure a central corneal thickness (CCT) value of the eye. In practical terms the light source of CCS emits a beam of (white) light towards cornea. A lens system is arranged on an optical path between the light source and the cornea. The lens system disperses light in such a way that different wavelengths of the light have different focal points. As the dispersed light reflects from the cornea a spectrum of the reflected light is measured. In the spectra, one can find, peak intensity values relating to different wavelengths of the light. For example, a first peak might be associated with a first wavelength and a second peak with a second wavelength. Difference between the first and the second wavelengths corresponds to a certain thickness value (depending on geometry and optical properties of the lens system), thus the thickness can be measured. CCS can be used to measure a corneal thickness. The corneal thickness value can be measured from any visible part of the eye surface. If the measurement is carried out from “central” portion of the cornea the measured value can be also referred as a central corneal thickness (CCT) value.

Furthermore, it will be appreciated that the confocal chromatic sensor can be used also to measure a displacement. The displacement refers to changes of distance from the sensor to the target (i.e for example from CCS to corneal surface). Resolution of measurement of a displacement of tens of nanometres can be obtained. Measurement frequency of tens of kilohertz (kHz) can be obtained. Measuring a time series of displacement values can be used to determine frequency of possible oscillations on the cornea. Optionally, the confocal chromatic sensor uses infrared light (IR) as a measuring beam. Beneficially, the IR light is not seen by the eye of the patient. In such a case, the temptation of the subject to blink the eye is reduced during the initial phase when setting a right relative position between the tonometer and the eye and during the measurement.

Optionally, the confocal chromatic sensor is operable to measure a distance between the cornea and the tonometer to provide an indication of positioning of the tonometer with respect to the eye. Optionally, the confocal chromatic sensor is used for measuring the distance between the confocal chromatic sensor and the eye from few millimetres to tens of millimetres. Optionally, the indication is provided as one of: a visual feedback or an acoustic feedback. For example, a measured distance between the tonometer and the cornea may be displayed on a screen of a device. Optionally, the visual feedback is represented in the form of a textual information or a graphical information. In case of the tonometer, which is handheld, the position of the tonometer may be optimized manually. Optionally, the measured distance is utilized as a feedback signal in an automatic position control system. Herein, a relative position of the tonometer with respect to the eye is optimized using at least one actuator. The at least one actuator is controlled using the measured distance as a feedback signal. Advantageously, the visual feedback or the acoustic feedback is used in order to assist the correct positioning of the tonometer with respect to the eye. Further optionally the indication can be used to initialize a measurement cycle automatically.

The term “controller” refers to a computational device that is operable for controlling the overall operation of the tonometer. The controller, in operation, performs tasks using the execution unit, using the measuring unit, using the at least one confocal chromatic sensor, responds and processes information. In an example, the controller may be an embedded microcontroller, a microprocessor, and the like. In this regard, the controller is coupled with the execution unit, the measuring unit and the at least one confocal chromatic sensor. The controller may be implemented as an internal component of the tonometer, an external component of the tonometer, or a combination thereof.

Optionally, the at least one measurement unit is a first confocal chromatic sensor or a second confocal chromatic sensor. The controller can be further configured to use the first confocal chromatic sensor and/or the second confocal chromatic sensor to measure an intraocular pressure value of an eye. This measurement can be done by configuring the first or the second confocal chromatic sensor to measure an oscillation frequency of free oscillations of the cornea caused by the impact (of the impact means) and to use the measured oscillation frequency to determine the intraocular pressure value of the eye. In this regard, the measuring unit includes the first or the second confocal chromatic sensor. It will be appreciated that the confocal chromatic sensor is placed at an appropriate position, for identifying the oscillations of the cornea accurately. Beneficially, the confocal chromatic sensor is customized to identify the frequency of oscillations of the cornea and to find the IOP value of the eye as a function of the frequency of the oscillations of the cornea. It will be appreciated that the confocal chromatic displacement sensors may conduct thousands of observations per second with a resolution of tens of nanometres and thus allows precise measurement of the amplitude of oscillation of at least one point of the cornea and thus use those observations to find the oscillation frequency. Optionally, the amplitude of oscillation of the at least one point of the cornea is used for determining the resonance frequency of the cornea. The resonance frequency of the cornea lies typically in a range of 100-500 Hz. Advantageously, the technical effect of using the first confocal chromatic sensor in the measuring unit is that the IOP value that the same confocal chromatic sensor which is used to measure thickness of the cornea can be also used to determine IOP by measurement of the oscillation frequencies. Benefit of having the second confocal chromatic sensor is that this way the second confocal chromatic sensor can be tailored (or configured) to provide as output IOP values instead of thickness values making the design more straightforward. As discussed the thickness value can be corneal thickness in general or for example a central corneal thickness.

Optionally, the at least one measuring unit is a velocity measurement unit, for measuring a velocity profile of the impact means, during the impact, and the controller is further configured to use the measured velocity profile to determine the intraocular pressure value of the eye. The velocity profile (such as speed as function of time=>acceleration) can be used to determine the intraocular pressure of the eye for example by using look up table or other predetermined formula. In this example the tonometer can be considered to be re-bound tonometer. It will be appreciated that the tonometer provides an accurate determination of the intraocular pressure value of the eye. In this regard, the incorporation of the velocity measurement unit in the tonometer enhances and provides more precise determination of the intraocular pressure. Further, measurement of the velocity profile of the impact means during the interaction with the cornea gives the tonometer valuable dynamic data, where the data is ingeniously utilized by the controller to refine the calculation of intraocular pressure with the help of look up table or predetermined formula. In this example the tonometer is a re-bound tonometer.

Particularly, the measurement of the accurate intraocular pressure value of the eye, the corneal thickness is also used as an important parameter, where it is evident that the thickness of the cornea is non-uniform over the whole cornea and depends on the location in both healthy and diseased cornea, and the corneal thickness is considered to influence on the accuracy of the IOP measurement. The CCS is used to measure the corneal thickness that varies from the central corneal thickness. Advantageously, incorporating the corneal thickness measurement in the same unit with the IOP measurement with impact means ensures that the corneal thickness is determined at the same location where the impact means was impacting the cornea and thus, correct corneal thickness reading can be used to correct the IOP result. Further, the controller takes this velocity profile and uses it ingeniously in determining the intraocular pressure value of the eye.

Furthermore, by incorporating the velocity profile into the intraocular pressure calculation, the tonometer gains a more precise understanding of the eye's response to the impact, which not only enhances the precision of the intraocular pressure measurement but also provides additional insight into the biomechanical properties of the eye during the impact. Further, measurement of the velocity profile of the impact means during the interaction with the cornea gives the tonometer valuable dynamic data, where the data is ingeniously utilized by the controller to refine the calculation of intraocular pressure with the help of look up table or predetermined formula.

Further optionally, when the impact means is an air impulse having a first duration of time and a first force, the measuring unit is a functional combination one of: the controller and the first confocal chromatic sensor or the controller and a second confocal chromatic sensor. I.e. the controller can receive information from the first confocal chromatic sensor (CCS) (i.e. same as was used to measure thickness value) or from the second confocal chromatic sensor (dedicated for measuring oscillations). The controller will then use this information (distance from the first or the second sensor in practical terms) to measure an oscillation frequency of free oscillations of the cornea caused, by the impact and to use the oscillation frequency to determine the intraocular pressure value of the eye. Term “a functional combination of” refers that data from CCS is processed by the controller and thus CCS and the controller together work as measurement means. In preferred implementation there is just the first CCS i.e. the first CCS is used for both thickness measurement and for measuring oscillations. Optionally the tonometer is an air impulse tonometer. Benefit of air impulse is that no physical object is in contact with eye during the measurement cycle. Further optionally measurement of an oscillation frequency of free oscillations of the cornea is started after the first duration of time. Technical benefit of this is that we can time oscillation measurements (thus measuring IOP) precisely to start when valve of the air nozzle is closed.

Optionally, when the first force of the air impulse is stopped, the decay of the force should be fast, preferably significantly shorter time than the time period of the free corneal oscillation. This way air impulse as such does not interfere with measurement, but eye can oscillate freely, due to lack of first force caused by the flow of air. Optionally, the controller is configured to correct the measured intraocular pressure value using the measured corneal thickness value (or central corneal thickness value). In this regard, the corneal thickness value of the cornea is a significant factor when determining IOP value of the eye. As an example, a thicker cornea may lead to false high measurement of the IOP value, and a thinner cornea may lead to false low measurement of the IOP value. Therefore, the IOP value is corrected based on the corneal thickness value of the eye or by the central corneal thickness value.

As an example, if the IOP is determined using re-bound tonometer principle the values of the measurement can be too low if the corneal thickness is thinner than normally. On the other hand, if the corneal thickness is thicker than normally the values might be too high.

As an example, a table I (below) values can be used as a basis for the correction calculation when a central corneal thickness is measured. For example, if the thickness is 485 micrometers then the IOP value is corrected by adding 3 mmHg. The table values should be modified if the measurement point is not central. The table can be used as look up table or, for example, a correction value as function of the corneal thickness can be derived from the values. Technical effect of this is that we can get more accurate measurements of intra ocular pressure.

TABLE I
Example of IOP value corrections as
function of central corneal thickness

| Cornea Thickness | Correction Values |
|---|---|
| (micrometers) | (mm Hg) |
| 465 | 4 |
| 485 | 3 |
| 505 | 2 |
| 525 | 1 |
| 545 | 0 |
| 565 | −1 |
| 585 | −2 |
| 605 | −3 |
| 625 | −4 |

As an other example the central corneal thickness value of the cornea is correlated to a resonance frequency of the cornea. The resonance frequency of the cornea is related to the IOP value of the eye as discussed. Therefore, the central corneal thickness value can be used for correcting relation between the resonance frequency and the IOP value, leading to an accurate determination of the IOP value of the eye. A table I above might be used for the correction. Similar table can be formed for the corneal thickness values which are measured from other parts than the central.

Notably, the confocal chromatic sensor measures the corneal thickness value of the eye as it possesses the ability to simultaneously measure a position of an external surface and an internal surface of the cornea. More optionally, the confocal chromatic sensor measures the position of the external surface and the internal surface of the cornea, simultaneously, in a condition when the cornea oscillates. The thickness of the cornea characterize the stiffness of the cornea, which is a disturbing factor when measuring the internal pressure of the eye. Moreover, when the thickness of the cornea is measured, its value may be used to apply a correction when calculating the internal pressure value of the eye. This allows to calculate the internal pressure of the eye with improved precision. Optionally, a correction factor for the IOP value is determined using regression analysis. Advantageously, the technical benefit of this is that the IOP value of the eye can be corrected precisely by taking into consideration the corneal thickness value of the eye.

Optionally, the at least one measuring unit is configured to measure the intraocular pressure value of an eye during a first period of time (dt). The first period of time starts at the first moment of time (t1) and the duration of the first period of time is until a second period of time (t2). The first moment of time (t1) is the moment of time when the impact means collide (impact) the cornea. The impact will cause the cornea to oscillate. Oscillations will damp over time and are virtually nonexistent at a second moment of time. In other words, the total duration of the first period of time is preferably at least duration of oscillations. Furthermore the first confocal chromatic sensor is configured to measure the corneal thickness value of the eye at a second moment of time (t2). In this regard, a difference between the first moment of time (t1) and at the second moment of time (t2) lies in a range of 1 to 3 seconds (could be 0.1, 0.5, 1.0, 1.5, 2.0, 2.5, 3.0 or up to 4.0 sec for example).

Optionally, the IOP value and the corneal thickness value of the eye is measured at the different moments of time so as to correct the IOP value measured during the first period of time (between t1 and t2) based on the corneal thickness value of the eye measured at the second moment of time (t2). As a first example, the IOP measured may be measured during first 0.1 sec from the impact of the impact means (i.e. 0.1 sec from t1), and the corneal thickness value may be measured later after 2 seconds of measuring the IOP value, at 2.1 seconds. Advantageously, the technical effect of measuring the IOP of an eye during first period of time (dt) and the corneal thickness value of the eye at least at a second moment of time (t2) is that the IOP value can be easily and/or accurately corrected based on the corneal thickness value of the eye. Alternatively, the corneal thickness value can be measured before the first period of time.

Optionally, the first period of time is a time difference (dt) between the first moment of time (t1) and the second moment (t2). The duration of the first period of time can be determined based on at least one of selected from: the measured corneal thickness value, or the measured intraocular pressure value. In this regard, the duration of the first period of time (dt) is a function of at least one of: the measured corneal thickness value, the measured intraocular pressure value. In an implementation, the duration of the first period of time (dt) may be determined based on the measured corneal thickness value. The thickness can have an impact on damping time constant related to the corneal oscillations. If the thickness is lower the oscitations might dampen faster in comparison if the thickness is higher. This is due to difference between elastic properties of the eye for lower corneal thickness value to higher values. In another implementation, the duration of the first period of time (dt) may be determined based on the measured IOP value. Optionally, the duration of the first period of time (dt) is calculated so as to ensure accurate correction of the measured intraocular pressure value based on the corneal thickness value. Advantageously, the technical effect of determining the duration of the first period of time is that corneal thickness value and/or the IOP value of the eye can be measured accurately between a given time interval and further, the IOP value can be accurately corrected based on the corneal thickness value. Indeed this arrangement ensures that, corneal thickness value measurement with confocal chromatic sensor is carried out when the eye surface is not oscillating. This way the measurement is more accurate. One optional way to determine duration of the first period of time is to measure a duration of the oscillations caused by impact. In practice to measure with the confocal chromatic sensor, amplitude of the oscillations as function of time after the impact. As soon as the amplitudes are insignificant, we can use that moment of time as the second moment of time (t2). As further technical effect for determining the duration of the first period of time is that we do not start a next measurement cycle (using impact means) before oscillations have settled down. This eliminates possible problems of interference of previous measurement with next measurement.

Optionally, the first period of time (t1) is before or after the second moment of time (t2). In this regard, the intraocular pressure value is measured before or after the corneal thickness value of the eye. Optionally, the intraocular pressure value and the corneal thickness value of the eye can be measured at a same instant. Advantageously, the technical effect of determining the time difference between the first moment of time (t1) and the second moment of time (t2) (i.e the duration of first period of time) is that corneal thickness value and/or the IOP value of the eye can be measured accurately between a given time interval and further, the IOP value can be accurately corrected based on the that corneal thickness value.

Optionally, the controller is configured to repeat the measurement of
the intraocular pressure value using the at least one measuring unit during a second time period, wherein the second time period is between third moment of time (t3) and a fourth moment of time (t4) and
the corneal thickness value using the first confocal chromatic sensor at least at the fourth moment of time (t4).

In this regard, the intraocular pressure value and the corneal thickness value is measured again during the second period of time (i.e. between the third moment of time (t3) and the forth moment of time (t4)). Optionally, a time difference between the third moment of time (t3) and the fourth moment of time (t4) lies in a range of 0.1 seconds. The third moment of time (t3) and the fourth moment of time (t4) occurs after the first moment of time (t1) and the second moment of time (t2). Referring to the first example, the intraocular pressure value may be measured at 5 seconds and the corneal thickness value may be measured at 7 seconds.

Optionally, the controller is further configured to repeat the determination (or measurement) of the intraocular pressure value using the at least one measuring unit and the corneal thickness value using the first confocal chromatic sensor until the corrected intraocular pressure value remains within predetermined tolerances. In other words, the measurement of the IOP value and the corneal thickness value of the eye is measured and corrected at varying instants of time. The measurement is continued until a corrected value of the IOP is within predetermined tolerances at different instants. Referring to the first example, the intraocular pressure value measured at t1 may be corrected based on the corneal thickness value measured at t2. Similarly, the intraocular pressure value measured at t3 may be corrected based on the corneal thickness value measured at t4, and so on. In said example, if, the IOP value at t1 is similar (within say +−5%) to IOP value at t3, the IOP value obtained at t1 and t3 may be considered as the IOP value of the eye. In said example, if the IOP value at t1 is different (difference more than 5%) than the IOP value at t3, the measurement of the IOP value and the corneal thickness value may be continued further at t4 and t6, respectively. Advantageously, repeating the measurement of the intraocular pressure value and the corneal thickness value significantly increases accuracy and precision in measurement of the IOP value of the eye. The predetermined tolerance can be for example 1.0 mmHg i.e if deviation of measurements is less than the predetermined tolerance the measurement cycles can be stopped.

Optionally, the tonometer is a rebound tonometer. In this regard, the term “rebound tonometer” refers to an instrument used for measuring the property of the object. In rebound tonometer IOP is measured by measuring with the at least one measuring unit velocity profile of the impact means as it is applied on a cornea of the eye and bounces back.

The present disclosure also relates to the method as described above. Various embodiments and variants disclosed above, with respect to the aforementioned first aspect, apply mutatis mutandis to the method.

Optionally, the measured corneal thickness value is used to correct the measured intraocular pressure value.

Optionally, the intraocular pressure value of an eye is measured during a first period of time), and the corneal thickness value of the eye is measured at least at a second moment of time (t2).

Optionally, the first period of time is a period of time difference (dt) between the first moment of time (t1) and second moment of time (t2) and is determined based on at least one of selected from the corneal thickness value or intraocular pressure value.

Optionally, the method further comprises repeating the steps (a) and (b) until the corrected intraocular pressure value within predetermined tolerance.

Optionally, the first period of time is before or after the second moment of time (t2).

Optionally the measurement of the oscillation frequency is started after the first duration of time i.e. after the air impulse has been stopped (such as within 0.1, 0.2, 0.3, 0.4, 0.5, 1, 2, 3, 4 or 5 millisecond after stopping).

As a summary a tonometer for determining properties of eye is presented. Properties which are determined (via measuring directly or indirectly) are intraocular pressure of the eye and central corneal thickness. The tonometer comprises an execution unit which is used to launch an impact means towards a cornea of the eye. The impact means can be for example a probe. At least on measuring unit is used to measure intraocular pressure. Again, as an example if the impact means was a probe, then the measuring unit is for measuring velocity profile of the probe during the impact. This velocity profile can be used to determine the intraocular pressure. The tonometer comprises also a first confocal chromatic sensor. The confocal chromatic sensor is used to measure (or determine) a corneal thickness value of the eye. Execution unit, measuring unit and the confocal chromatic sensor are all controlled (or used) by a controller. The presented setup enables reliable and accurate tonometer for determining the said properties.

According to other embodiment the impact means is an air impulse. In this embodiment the measurement unit is a functional combination of controller and a first confocal chromatic sensor. The combination is configured to measure oscillations and derive from those the intra ocular pressure of eye. The pressure reading is corrected using corneal thickness measurement done with the first confocal chromatic sensor. Alternatively, the functional combination can comprise a combination of the controller and the second confocal chromatic sensor. Benefit of having two different confocal chromatic sensors is that the first one can be tuned to measure thickness values (sub-micrometer accuracy) and other one to measure corneal movements (micrometer accuracy).

There are several technical benefits in combining rebound tonometer to CCS pachymeter and correcting the IOP value with corneal thickness value. Firstly cornea thickness is not uniform. The thickness of the cornea varies depending on the location in both healthy and diseased cornea, and corneal thickness is considered to influence on the accuracy of the IOP measurement. Usually the central corneal thickness (CCT) value is used to correct the IOP readings in applanation tonometry. In rebound tonometry, the measurement (that is the probe impact) may not always hit to the center of the cornea. In these regions, CCS can be used to measure corneal thickness that varies from the central corneal thickness (CCT). One special case here is astigmatism, where the corneal thickness at different locations can vary significantly. This way we know impact of the thickness on the measurement result as CCS can be configured to measure thickness at the spot of the impact.

Furthermore corneal thickness exhibits fast changes. Use of anesthetic drops alters the corneal thickness in the short term. These drops are often needed during rebound IOP measurement with patients who suffer from corneal injury since otherwise the measurement would be painful for them. The IOP itself can modify the corneal thickness so that at higher pressures the cornea stretches and narrows compared to lower pressure levels. This can happen in some minutes, for example, the IOP can vary up to 5 mmHg depending on whether the person is standing/walking or sitting/relaxing. Also, tensing shoulders can increase the IOP up to 5 mmHg.

Also, supine body position has an effect on central corneal thickness (CCT). I.e. the central corenal thickness can change rapidly thus leading to error measurement results on IOP if the corneal thickness is not known.

In addition, corneal thickness exhibits slow changes. The normal use of therapeutic contact lenses or the misuse of traditional contact lenses is known to lead to corneal swelling, in other words changing the corneal thickness value. Intensive myopization among children can change the corneal thickness even in a few months. Fast progressing keratoconus can change the corneal thickness even in a few months.

For above reasons, it is important to measure corneal thickness at the same time as the IOP measurement.

### DETAILED DESCRIPTION OF THE DRAWINGS

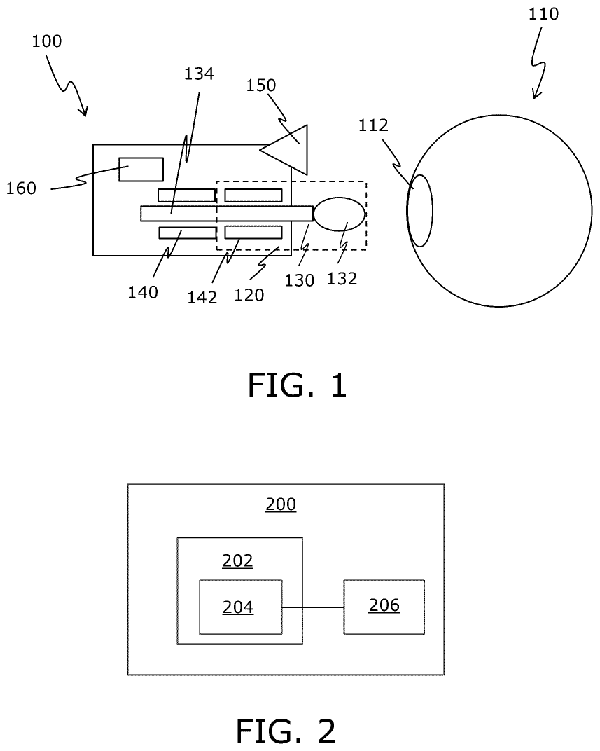

Referring to FIG. 1, illustrated is a schematic illustration of a tonometer 100 for measuring (or determining) properties of an eye 110, in accordance with an embodiment of the present disclosure. The tonometer 100 comprising an execution unit 120 comprising an impact means 130 arranged to apply an impact on a cornea 112 of an eye 110, at least one measuring unit (depicted for example as a measuring unit 140), at least one confocal chromatic sensor (depicted for example as a confocal chromatic sensor 150), a controller 160 connected to the execution unit 120, the measuring unit 140 and the confocal chromatic sensor 150.

According to one example the impact means 130 is a probe comprising elongated magnetic body 134 and a tip part 132 connected on a first end of the magnetic body 134. When in use the controller 160 provides electricity via a set of electrical loops 142, thus creating a magnetic force to the elongated magnetic body 134. The magnetic force ejects the impact means 130 towards the cornea 112 of the eye 110. The tip part 132 of the probe hits the cornea 112 of the eye at first moment of time (t1) and the probe bounces back inside of the tonometer. During the movement the elongated magnetic body 134 of the impact means 130 (the probe) induces electric current to the measurement unit 140. The measurement unit 140 in the present example is a measurement coil (arranged to surround at least partly the elongated magnetic body 134 of the impact means 130. This induced electric current is a function of velocity of the impact means 130 during the impact (and before and after the impact). The velocity of the impact means can be used to determine the intraocular pressure of the eye. Indeed, if change of speed (dv/dt (derivate of velocity in respect to time)) of the impact means is high it indicates higher intraocular pressure (eye is stiff) than if the change of speed is lower. The determination can be done using for example lookup table comprising speed change vs intraocular pressure (IOP) values or using a formula in which IOP is function of (dv/dt). This way the intraocular pressure of the eye can be determined (measured).

The impact means will cause the cornea 112 to oscillate for first period of time (t1 to t2). After the oscillations have been stopped (or come near to stop) the confocal chromatic sensor 150 is used to measure central thickness of the cornea 112. The central thickness value of the cornea can be used to correct (calibrate, adjust) the measured intraocular pressure value using for example lookup table or correlation formula.

FIG. 1 is merely an example, which should not unduly limit the scope of the claims herein. A person skilled in the art will recognize many variations, alternatives, and modifications of embodiments of the present disclosure.

Referring to FIG. 2, illustrated is a block diagram illustrating a tonometer 200 for measuring properties of an eye, in accordance with an embodiment of the present disclosure. The tonometer 200 comprises at least one confocal chromatic sensor (depicted for example, as a confocal chromatic sensor 204) comprised in a measuring unit 202 and a controller 206 configured to use the confocal chromatic sensor 204 to measure an intraocular pressure value of an eye.

FIG. 2 is merely an example, which should not unduly limit the scope of the claims herein. A person skilled in the art will recognize many variations, alternatives, and modifications of embodiments of the present disclosure.

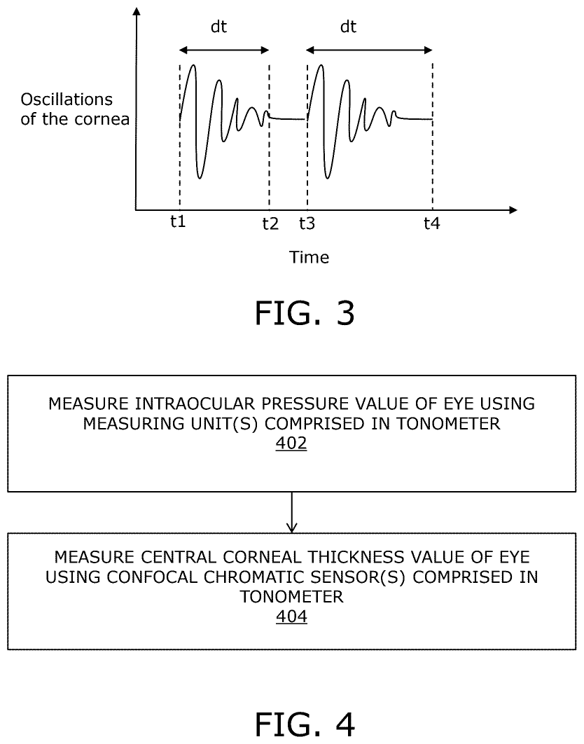

Referring to FIG. 3, illustrated is a graphical representation depicting oscillations in cornea with respect to time, in accordance with an embodiment of the present disclosure. The oscillations in the cornea are represented on Y-axis and the time is represented on X-axis. The oscillations in the cornea are depicted as damped oscillations at a first moment of time (t1), at a second moment of time (t2), at a third moment of time (t3), and at a fourth moment of time (t4). Oscillations start (as discussed above) upon impact of the impact means to the cornea of the eye. The cornea oscillates with an oscillation frequency of free oscillations of the cornea for a first period of time (time between t1 to t2 or t3 to t4). The oscillations dampen and at second or fourth moment of time corneal thickness value can be measured. In one embodiment the measurement can take place during a measurement period between the second moment of time (t2) and third moment of time (t3). Depending on setup the measuring unit can be the first confocal sensor in which case the measuring unit is measures oscillations, determines frequency of oscillations and based on that determines measured IOP value. In alternative setup the measuring unit is part of rebound tonometer i.e. a part which measures velocity of the impact means and uses that as indirect measurement of the IOP as discussed. Oscillation frequency can be measured with confocal chromatic sensor 150 of the FIG. 1. This oscillation frequency can be further used to determine IOP with use of lookup table or equation.

FIG. 3 is merely an example, which should not unduly limit the scope of the claims herein. A person skilled in the art will recognize many variations, alternatives, and modifications of embodiments of the present disclosure.

Referring to FIG. 4, illustrated is a flowchart depicting steps of a method for measuring properties of an eye, in accordance with an embodiment of the present disclosure. At step 402, an impact is applied on a cornea of the eye using an execution unit comprised in a tonometer, wherein the execution unit comprises an impact means arranged to apply, when in use, the impact on the cornea of the eye. At step 404, an intraocular pressure value of the eye is measured using at least one measuring unit comprised in the tonometer. At step 406, a corneal thickness value of the eye is measured using at least one confocal chromatic sensor comprised in the tonometer, wherein the at least one measuring unit impact means, during the impact, and wherein the measured velocity profile is used to determine the intraocular pressure value of the eye.

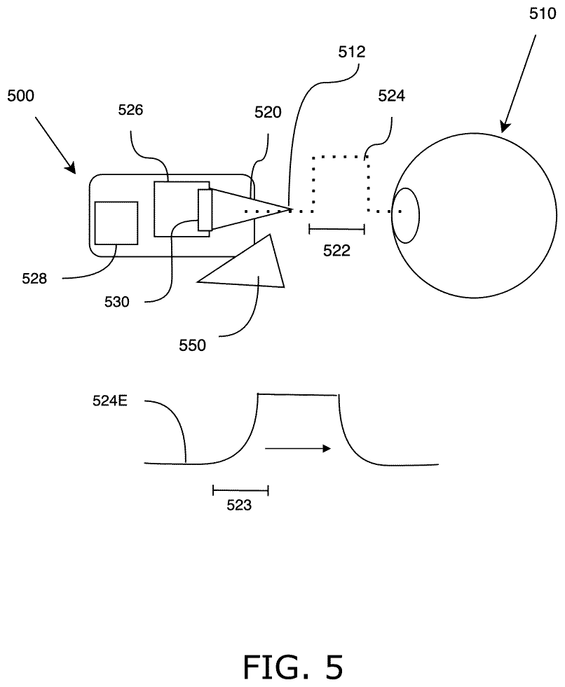

FIG. 5 is an illustration of a tonometer 500, wherein impact means is an air impulse 524, (enlargement of typical pulse form is in 524E). The air impulse 524, 524E has a first force (illustrated as height in y-axis direction) and a first duration 522. The air impulse is created by opening a valve 530 between a nozzle 520 and pressure chamber 526 using control signal from a controller 528. The air escapes from opening 512 of the nozzle 520 towards eye 510. The first confocal chromatic sensor 550 measures central corneal thickness as well as oscillations resulting on the air impulse. Measuring unit is a functional combination of the controller 528 and the confocal chromatic sensor 550 which measures displacement caused by the air impact (i.e. oscillations). Enlargement 524E of the air impulse illustrates a decay time 523 after the valve 530 have been shut down. The decay time 523 is in range of 2 msec i.e. shorten than a single oscillation of the oscillations. This way we ensure free oscillations which are not disturbed by air impulse. In the example figure the air impulse 524, 524E is travelling from the tonometer 500 towards the eye 510 as indicated with an arrow. A right side edge of the air impulse impacts 524, 524E eye surface first, followed by a first force and the air impulse end having the decay time 523 is indicated as left edge of the air impulse. The first force can be constant, or it can vary. As an example, it can start from low value and increase after that.

The aforementioned steps are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A tonometer for determining properties of an eye, the tonometer comprising
an execution unit comprising an impact means arranged to apply, when in use, an impact on a cornea of the eye;
at least one measuring unit,
a first confocal chromatic sensor;
a controller connected to the execution unit, the measuring unit and the first confocal chromatic sensor, wherein the controller is configured, when in use, to use:
the execution unit to apply an impact on the cornea of the eye using the impact means;
the at least one measuring unit to determine an intraocular pressure value of the eye; and
the first confocal chromatic sensor to measure a corneal thickness value of the eye.

2. The tonometer according to claim 1, wherein the at least one measuring unit is a velocity measurement unit, for measuring a velocity profile of the impact means, during the impact, and the controller is further configured to use the measured velocity profile to determine the intraocular pressure value of the eye.

3. The tonometer according to claim 1, wherein the impact means is an air impulse having a first duration of time and a first force, and wherein the measuring unit is a functional combination one of: the controller and the first confocal chromatic sensor or the controller and a second confocal chromatic sensor, wherein the controller is further configured to use one of: the first confocal chromatic sensor or the second confocal chromatic sensor to measure an oscillation frequency of free oscillations of the cornea caused, by the impact and to use the oscillation frequency to determine the intraocular pressure value of the eye.

4. The tonometer according to claim 1, wherein the controller is further configured to use the first or a second confocal chromatic sensor to measure an oscillation frequency of free oscillations of the cornea caused, by the impact and to use the oscillation frequency to determine the intraocular pressure value of the eye.

5. The tonometer according to claim 1, wherein the controller (160, 206) is configured to correct the measured intraocular pressure value using the measured corneal thickness value.

6. The tonometer according to claim 1,
wherein the at least one measuring unit is configured to measure the intraocular pressure value of an eye during a first period of time, and
the first confocal chromatic sensor is configured to measure the corneal thickness value of the eye at a second moment of time.

7. The tonometer according to claim 6, wherein the first period of time is a period of time between the first moment of time and the second moment of time, and is determined based on at least one of selected from: the measured corneal thickness value, or the measured intraocular pressure value.

8. The tonometer according to claim 6, wherein the first period of time is before or after the second moment of time.

9. The tonometer according to claim 6, wherein the controller (160, 206) is configured to repeat the measurement of
the intraocular pressure value using the at least one measuring unit during a second time period, wherein the second time period is between third moment of time and a fourth moment of time and
the corneal thickness value using the first confocal chromatic sensor at least at the fourth moment of time.

10. The tonometer according to claim 1, wherein the controller is further configured to repeat the determination of the intraocular pressure value using the at least one measuring unit and the corneal thickness value using the at least one confocal chromatic sensor until the corrected intraocular pressure value remains within predetermined tolerance.

11. The tonometer according to claim 1, wherein the tonometer is a rebound tonometer.

12. The tonometer according to claim 1, wherein the tonometer is an air impulse tonometer.

13. The tonometer according to claim 3 wherein measurement of an oscillation frequency of free oscillations of the cornea is started after the first duration of time.

14. The tonometer according to claim 3, wherein the first force is stopped with a decay time that is shorter than the time period of the free corneal oscillation.

15. A method of measuring properties of an eye, the method comprising:
a) applying an impact on a cornea of the eye using an execution unit comprised in a tonometer, wherein the execution unit comprises an impact means arranged to apply, when in use, the impact on the cornea of the eye;
b) measuring an intraocular pressure value of the eye using at least one measuring unit comprised in the tonometer and
c) measuring a corneal thickness value of the eye using at least one confocal chromatic sensor comprised in a tonometer.

16. The method according to claim 15, wherein the at least one measuring unit is a velocity measurement unit, for measuring a velocity profile of the impact means, during the impact, and wherein the measured velocity profile is used to determine the intraocular pressure value of the eye.

17. A method according to claim 15, wherein the impact means is an air impulse having a first duration and a first force, and wherein the at least one measuring unit is a functional combination of the controller and the first confocal chromatic sensor or a second confocal chromatic sensor, wherein the controller is further configured to use the first confocal chromatic sensor or the second confocal chromatic sensor to measure an oscillation frequency of free oscillations of the cornea caused, by the impact and to use the oscillation frequency to determine the intraocular pressure value of the eye.

18. The method according to claim 15, wherein
the measured corneal thickness value is used to correct the measured intraocular pressure value.

19. The method according to claim 15, wherein
the intraocular pressure value of the eye is measured during a first period of time, and
the corneal thickness value of the eye is measured at least at a second moment of time.

20. The method according to claim 19, wherein the first period of time is a period of time difference between a first moment of time and a second moment of time and is determined based on at least one of selected from the corneal thickness value or intraocular pressure value.

21. The method according to claim 15, further comprising repeating the steps (a) and (b) until the corrected intraocular pressure value remains within predetermined tolerance.

22. The method according to claim 19, wherein the first period of time is before or after the second moment of time.

23. The method according to claim 15, wherein the at least one measurement unit is a rebound tonometer.

24. The method according to claim 15, wherein the at least one measurement unit is an air impulse tonometer.

25. The method according to claim 17 wherein the measurement of an oscillation frequency of free oscillations of the cornea is started after the first duration of time.

26. The method according to claim 17, wherein the first force of the air impulse is stopped with a decay time that is shorter than the time period of the free corneal oscillation.
